(19) United States
(12) Patent Application Publication
(10) Pub. No.: US 2005/0069513 A1
(43) Pub. Date: Mar. 31, 2005

(54) Amphiphilic compounds for pharmaceutical or cosmetic use

(75) Inventor: Nicolas Calvet, Saint Laurent du Var (FR)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Physica S.A.R.L., Vallauris (FR)

(21) Appl. No.: 10/499,542

(22) PCT Filed: Dec. 24, 2002
(86) PCT No.: PCT/FR02/04561

(30) Foreign Application Priority Data
Dec. 27, 2001 (FR) 01 16936

(51) Int. Cl. A61K 38/00; A61K 7/06; A61K 7/11

(52) U.S. Cl. 424/70.14; 514/2

(57) ABSTRACT

The invention relates to amphiphilic compounds, for pharmaceutical or cosmetic use, consisting of an ion pair complex between an acylamino acid and a biologically active molecule. Said formed compounds enable the enhancement of the properties of pharmaceutical or cosmetic molecules, in particular of small organic molecules, peptides and proteins, nucleotides or genes.

### TECHNICAL FIELD

The present invention relates to the formation and cosmetic and pharmaceutical use of an amphiphilic compound known as an “amphiphilic ion pair” (or “AIP”) resulting from ionic interaction between an acylamino acid and a pharmaceutical or cosmetic active molecule.

### BACKGROUND ART

The pharmacological activity of an active principle depends not only on its chemical structure and therefore its physico-chemical properties, but also on its capacity to join its active site, i.e. the location where it acts in a sufficiently large quantity. This property is known as “bioavailability”.

The bioavailability of an active principle largely depends on the route used for its administration (enteral or parenteral route). Indeed, depending on the route of administration, the active principle can encounter obstacles which reduce its absorption; it can be dissolved to a greater or lesser extent in biological liquids, undergo a greater or lesser number of biotransformations and finally be absorbed to a greater or lesser extent. Also, it is important that the best route of administration is chosen for a given active principle.

The most common route of administration is the oral route. Ease of swallowing a medicine and the fact that the digestive tract is a favourable absorption site evidently account for this choice.

However, the use of the oral route as the preferred route of administration means that a significant number of potentially pharmalogically advantageous active molecules are ruled out as a result of biotransformation problems or lack of absorption. This would lead, indeed, to the use of increased doses of actives, engendering higher treatment costs and/or increased toxicity, meaning that the product could not be put on the market. The clinician may therefore resort to an alternative administration method, such as the injectable route (SC, IM, IV).

The person skilled in the art has thus sought technical means for enhancing the protection of active molecules, i.e. increasing the quantity of compounds capable of arriving at the absorption site. He has also sought to alter the resorption of active molecules by acting either on the solubility thereof in the case where dissolution is the factor slowing down the molecule's absorption, or by improving intestinal transmembrane passage.

With regard to improving absorption by lessening the number of bio-transformation phenomena, the person skilled in the art has developped mainly liquid preparations such as water-in-oil emulsions, i.e. systems wherein the the hydrophilic phase is dispersed in the lipophilic phase, or multiple emulsions of the Water-in-Oil-in-Water type.

United States patent U.S. Pat. No. 5,897,876 proposes H/L-type emulsified systems, i.e. made up of a hydrophilic phase dispersed in a lipophilic phase. These systems create a lipophilic environment around the active molecule, thus enabling the quantity of resorbed molecules to be increased. Indeed, they enable the protection of active principles against the destabilising action of proteolytic enzymes and gastrointestinal fluids. This protection thereby increases the quantity of active principle arriving at its absorption site.

However, the reduced proportion of the dispersed phase (lower than 10%) of these systems limits the quantity of active principle able to be solubilised. Furthermore, the presence of ethanol in the hydrophilic part can lead to destabilisation of the system and engender irritant effects at cell level.

A more recent approach consists in improving absorption by increasing the lipophilic character of the active molecules through the formation of ionic complexes. Patent U.S. Pat. No. 5,853,740 highlights the advantage of systems using sodium dodecylsulphonate as a complexing agent. These means are used in order to improve the absorption of active molecules which are only slightly resorbed (i.e. molecules only slightly absorbed at intestinal membrane level).

These complexes are made up of an ion pairing between the active molecules of an acidic or alkaline nature and an amphiphilic compound with an alkaline or acidic free ionic function. The complex resulting from this association comes either in the form of a precipitate in the hydrophilic phase (patent WO 0132218) or in dissolved form in an organic solvent (patents U.S. Pat. No. 5,770,559 and U.S. Pat. No. 5,853,740) of the ethanol, octanol, DMSO, DMF or N methyl pyrrolidone type.

One of the advantages of these complexes is that they allow the preservation of the three dimensional structure of the active substance. Certain molecules can indeed lose their biological activity in the event of the denaturation of this structure. These various patents indicate that the use of such complexes can improve the bioavailablility of active principles.

The authors of patent U.S. Pat. No. 5,770,559 use a method of preparing a homogeneous organic solution of an active compound in which said active compound is not normally soluble. This solubilisation is achieved by forming a hydrophobic ion pair complex between an amphiphilic compound and said active molecule. The complex thus obtained can be transformed into solid particles by precipitation in a supercritical fluid, for administrations by inhalation, notably using the nasal mucosa.

As with the preceding patent, patent WO 0132218 seeks to improve the bioavailablility of hydrophilic active principles which are insoluble in a lipophilic phase, by decreasing their solubility in a hydrophilic phase and by increasing their solubility in said lipophilic phase. To achieve this, the authors of said patent use amphiphilic counter-ions by forming an ion pair-type hydrophobic complex with the hydrophilic compound.

The inventions relating to these patents are aimed at reducing the hydrophilic character of the active principle. The approach of these patents is based on decreasing aqueous phase solubility in order to increase organic or lipophilic phase solubility. This aim is achieved through the formation of a hydrophobic ion pair complex. This formation results from the complexation between an active principle and an amphiphilic compound, notably sodium lauryl sulphate (SLS), sodium dodecyl sulphate (SDS) or a zwitterion.

The teaching of these patents is thus limited to the formation of hydrophobic ion pair complexes. These complexes are therefore essentially solubilised in organic solvents. These hydrophobic complexes can thus only be used with difficulty in a hydrophilic phase or in a dispersed phase of the hydrophilic phase in lipophilic phase-type, notably water in oil.

Means of obtaining inorganic salts (notably sodium or potassium) of acylamino acid have been known to the person skilled in the art since the 1950's. Due to their amphiphilic properties, these compounds have been used since this period in the field of detergents. Acylamino acids are also used in the field of cosmetics as amino acid biovectors.

More recently, acylamino acids have been used as absorption promoters. Patent U.S. Pat. No. 5,650,386 and patents WO 0135998 and WO 0151454 disclose the use of acylamino acids in pharmaceutical compositions.

Patent U.S. Pat. No. 5,650,386 describes a technique for encapsulating active principles. During preparation of the composition, the acylamino acids form microspheres constituting hollow matrices inside which the active principle is contained (or encapsulated). The active priniciple is thus protected against various forms of degradation.

Patent WO 0135998 describes acylamino acids as absorption promoting agents, dissolved in liposome-type lipidic biphasic vesicles.

Patent WO 0151454 describes an aqueous phase mixture between an acylamino acid and an active principle. In this patent, the mere presence of acylamino acids favours absorption.

All of the previously described systems only provide incomplete solutions in the case of ionisable hydrophilic and low-permeable molecules. Also, no teaching taken from the prior art has advocated the use of acylamino acids to form hydrophilic phase-soluble, ion pair complexes.

### DISCLOSURE OF THE INVENTION

The aim of the present invention is thus the formation of complexes between a compound and an active principle, which are hydrophilic phase-soluble and thus can be used in aqueous solution directly or in a H/L type dispersed system (hydrophilic phase dispersed in a lipophilic phase).

More precisely, the object of the present invention relates to a compound for pharmaceutical or cosmetic use, formed from an ion pair complex between an acylamino acid and a biologically active molecule, used in therapeutic or cosmetic treatments, the complex being amphiphilic.

The interaction between the acylamino acid and the pharmaceutical or cosmetic active molecule corresponds to the interactions found within the complexes, also known as coordination compounds. Such interaction is called coordination bond, coordinate bond or even dative bond according to the literature. The compound resulting from this association has amphiphilic properties and is thus known as “amphiphilic ion pair” (or “AIP”), or amphiphilic ion pair complex (or “AIP” complex).

The invention generally applies to molecules of the organic and hydrophilic type, the properties of which do not allow them to easily pass through the biological membranes, and/or which are rapidly bio-transformed in the body.

These amphiphilic compounds have the advantage of improving the availability of active principles in the body and preserving the 3D structure of these active priniciples. They also have the advantage of being inserted at the interfaces of dispersed systems, thus enabling the protection of said compound of bio-transformations and extending the lifetime of the active molecules as a result thereof.

### DETAILED DESCRIPTION OF THE INVENTION

More precisely, the invention applies to biologically active molecules such as short peptides, polypeptides, proteins, hormones, antigens, nucleotides or genes, the properties of which do not allow them to easily pass through the biological membranes, and/or which are rapidly bio-transformed in the body. According to the invention, an “AIP” complex is formed between one of these molecules and an acylamino acid. The absorption of this molecule will thus be improved due to the amphiphilic properties of this complex.

The principal advantage in these compounds is that the presence of the acylamino acid gives them an amphiphilic character and not a hydrophobic character. The “AIP” resulting from this association remains in solution and is preferably located at the interfaces of the dispersed systems. This surprising property distinguishes complexes formed from an acylamino acid from other complexes widely formed from ions, such as sodium lauryl sulphate (SLS) or sodium dodecyl sulphate (SDS), and having a hydrophobic character.

Thus, these complexes are preferably used for hydrophilic biologically active molecules. Indeed, the latter have difficulty in passing through the biological membranes due to their hydrophilic property. The amphiphilic nature of the complex thus improves the transmembrane passage of the active principle. Furthermore, this amphiphilic nature allows also the complex to be used in solution in water or in the hydrophilic phase of a dispersed system, which is impossible with a hydrophobic complex. As such, although the transmembrane passage of the active principle is improved, it can thus still be used in hydrophilic phases.

It should be noted that in forming a complex between an acylamino acid in its native state, i.e. non-salified, and an active principle which is insoluble in water, an amphiphilic complex which can then be solubilised in a hydrophilic phase and still passes easily through the biological membranes is also obtained.

The term acylamino acid refers to any compound resulting from an acylation between a fatty acid of natural, synthetic or modified origin and a natural, synthetic or modified amino acid, the fatty acid comprising 4-40 carbon atoms and the amino acid having at least one acid function and at least one free amine function. More particularly, one of the amine functions is located in the alpha position in relation to the carboxylic acid function.

The following can be used as amino acids for example: aspartic acid, glutamic acid, alanine, arginine, cysteine, glycine, histidine, isoleucine, leucine, lysine, methionine, phenylalanine, proline, ornithine, taurine, threonine, tryptophan, tyrosine, serine or valine.

The following can be used as fatty acids for example: capric acid, caprylic acid, lauric acid, myristic acid, palmitic acid, stearic acid, oleic acid, linoleic acid, linolenic acid, arachidic acid, essential fatty acids such as eicosapentaenoic acid (EPA), or docosahexaenoic acid (DHA).

Various acylamino acids are thus obtained, such as oleyl glycine, capryloyl glycine, oleyl sarcosine, lauroyl proline, oleoyl lysine, lauroyl lysine, palmitoyl phenylalanine or capryloyl serine.

According to a preferred embodiment, the amphiphilic compound results from the interaction between at least one of the reactive functions carried by the active principle and at least one reactive function of the acylamino acid, generally an acid or amine function. The reactive function of the active principle is generally alkaline or acidic. This is an interaction such as previously defined in the present application, i.e. of the type of those occurring within the complexes.

The term biologically active molecules, also widely known as active principles, relates to molecules having therapeutic or cosmetic properties. These biologically active molecules can be organic molecules, short peptides made up of 2-20 amino acids, nucleotides, genes, polypeptides, proteins, hormones or antigens.

The following can be cited as examples: amoxicillin, losartan, pravastatin, diclofenac, lidocaine, vancomicin, spiramicine, neomicin, colistine, cimetidine, ranitidine, insulin, vasopressin, calcitonin, angiotensin, secretin, heparin, growth hormone, erythropoietin, parathyroid hormone or filgastrine.

According to a preferred preparation method, said amphiphilic compounds can be inserted into dispersed systems. The term dispersed systems refers to systems consisting of two immiscible phases, one generally lipophilic and the other hydrophilic, wherein one or the other of these two phases makes up the dispersing phase or the dispersed phase. The term miscibility refers to the property whereby two compounds can be mixed together, forming a single continuous phase.

Thus, another object of the invention is a dispersed system comprising a complex according to the present invention.

According to a preferred embodiment, the internal dispersed phase (hydrophilic or lipophilic) and the dispersing phase (lipophilic or hydrophilic) contain one or more emulsifying and/or thickening agents.

According to a particularly advantageous embodiment of the invention, the ion pair complex is obtained by mixing two phases, A and B, which are prepared separately. Phase A contains at least one acylamino acid in the dissolved or dispersed state and forms a mixture which is miscible or dispersible in phase B. Phase B contains at least one active principle in the dissolved or dispersed state and forms a mixture which is miscible or dispersible in phase A.

During the incorporation of phase A into phase B, or vice versa, interaction commences between the acylamino acid and the active principle such that an “amphiphilic ion pair” is formed. When phase A is miscible in phase B, a single phase comprising the amphiphilic ion pair complex solubilised in this single phase is obtained after mixing. When phase A is dispersible in phase B, a dispersed system is obtained after mixing, in which the “AIP” complex is in solubilised form inside the internal phase and is preferably inserted at the interfaces of the dispersed system.

According to a preferred preparation method, at least the acylamino acid or the biologically active molecule is in native form. This means that either the acylamino acid and the biologically active molecule are both in native form, or one of them is in native form and the other is in the salt state.

It is also possible to isolate said amphiphilic compound in order to use it in various therapeutic compostions (tablets, gelatin capsules).

The examples given below are in no way limiting. The three first examples relate to methods of obtaining an amphiphilic complex according to the present invention. The fourth example is a permeation study of a compound according to the present invention.

### EXAMPLE 1

### Formation of an Ion Pair Between a Non-Salified Hydrophilic Active Principle (Calcitonin) and the Non-Salified Lipoamino Acid (Oleyl Methyl Glycine)

Two aqueous solutions are used. The first contains calcitonin. Due to the fact that the calcitonin is hydrophilic, the solution is clear. The second solution contains oleyl methyl glycine dispersed in the aqueous phase, the oleyl methyl glycine being only slightly soluble in water. This second solution has a milky white appearance.

After mixing the first and the second solution, a clear solution having a single phase is obtained. The obtained complex, calcitonin oleyl methyl glycine, is therefore amphiphilic.

### EXAMPLE 2

### Formation of an Ion Pair Between a Non-Salified Lipophilic Active Principle (Lidocaine) and the Non-Salified Acylamino Acid (Oleyl Glycine)

Two aqueous solutions are used. The first contains lidocaine dispersed in the aqueous solution. The second solution contains oleyl glycine dispersed in the aqueous phase, the oleyl glycine being only slightly soluble in water. This second solution has a milky white appearance.

After mixing the first and the second solution, both non-clear, a clear solution having a single phase is obtained. The obtained complex, lidocaine oleyl glycine, is therefore amphiphilic.

### EXAMPLE 3

### Formation of an Ion Pair Between a Salified Active Principle (Polymyxin E Sulphate) and the Non-Salified Acylamino Acid (Linoleyl Glycine).

A solution of NaOH in the quantity required for causing precipitation of all of the polymyxin E by deplacement of the sulphate salt is added to a solution of polymyxin E sulphate. Polymyxin E, also known as colistin, thereby goes back to its non-salified, basic structure. The resulting solution, solution B, is thus an aqueous phase with a precipitate of polymyxin E.

An aqueous solution, solution A, is then used which includes linoleyl glycine dispersed in solution A. Due to the fact that linoleyl glycine is only slightly soluble in water, solution A has a milky white appearance.

Solution A is added to solution B, the solution obtained having only a single clear phase. The polymyxin is thus resolubilized in the form of an amphiphilic complex, polymyxin E linoleyl glycine.

It should be noted that during the first step, the active principle being salified with a negative ion, the sulphate ion, the precipitation of the active priniciple in its non-salified form is obtained using a soda solution. In the case where the active principle is salified with a positive ion, the precipitation of the active principle in its non-salified form is obtained using a hydrochloric acid solution.

### EXAMPLE 4

### In vitro Study of Diffusion Through a Synthetic Membrane

An in vitro diffusion study is carried out with the aim of testing how well polymyxin E passes through a synthetic membrane. This membrane, of nylon type, is impregnated with lipid substances, in order to simulate diffusion through the intestinal lipid membrane. This study compares the performances of polymyxin E in the form of an ion pairing with an acylamino acid (oleyl methyl glycine) and in sulphate salified form.

The in vitro permeation study is carried out using Frantz cells. These cells have a donor compartment, in which a formulation containing polymyxin E in ion pair form or a polymyxin E sulphate solution is deposited, and a receptor compartment containing demineralised water. The measurements are carried out in 3 cells at the same time, for each of the forms of the polymyxin E (polymyxin E sulphate or polymyxin E oleyl methyl glycine). The two compartments are separated by the synthetic membrane.

The measurement of the quantity of polymyxin E which diffuses through the membrane is carried out by UV after: 1 hour, 2.5 hours, 4 hours, 6 hours and finally 7 hours.

The table below relates to a quantity of polymyxin E which diffused over the period of time; the values shown are cumulative values.

| | 0 h | 1 h | 2.5 h | 4 h | 6 h | 7 h |
|---|---|---|---|---|---|---|
| AIP of polymyxin E- | 0 | 0.80 | 2.75 | 4.05 | 5.17 | 6.25 |
| oleyl methyl glycine | | | | | | |
| (mg/l) | | | | | | |
| Aqueous solution of | 0 | 0.26 | 1.45 | 1.93 | 2.42 | 2.96 |
| polymyxin E sulphate | | | | | | |
| (mg/l) | | | | | | |

These results show that the ion pair Polymxyin E-oleyl methyl glycine diffuses 2 times more rapidly through a lipid synthetic membrane than in its sulphate-salified form.

1. A compound for pharmaceutical or cosmetic use comprising an ion pair complex between an acylamino acid and a biologically active molecules useful for therapeutic or cosmetic treatments, said complex being amphiphilic.

2. The compound according to Claim 1, wherein said biologically active molecule is hydrophilic and non-amphiphilic.

3. The compound according to claim 1, wherein said biologically active molecule is not soluble in water.

4. The compound according to claim 1, wherein said acylamino acid results from the condensation between a fatty acid of natural, synthetic or modified origin and a natural synthetic or modified amino acid.

5. The compound according to claim 4, wherein said amino acid comprises at least on amine function in the alpha position in relation to the carboxylic acid function of said amino acid.

6. The compound according to claim 1, formed from an ion pair complex between at least one reactive function of the acylamino acid and at least one reactive function of said biologically active molecule.

7. A dispersed system in which the internal dispersed phase is hydrophilic and comprises at least one amphiphilic compound according to claim 1 and in which the dispersing phase is lipophilic.

8. The dispersed system according to claim 7, wherein the internal dispersed phase and the dispersing phase contain one or more emulsifying and/or thickening agents.

9. A method of obtaining a compound according to claim 1 comprising the following steps:
separately preparing a first and a second phase, the first and second phase being miscible or dispersible one in the other, said first phase containing at least one acylamino acid in the dissolved or dispersed state and said second phase containing at least one biologically active molecule in the dissolved or dispersed state, and
mixing the first phase and the second phase.

10. The method according to claim 9, wherein said acylamino acid and said biologically active molecule are either both in native form, or one is in native form, and the other in salt form.

11. The compound according to claim 2 wherein said biologically active molecule is an organic molecule, a short peptide, a protein, an antigen, a nucleotide or a hormone.

12. The compound according to claim 6 wherein the reactive function of said biologically active molecule is an acid or amine function.
